(19) United States
(12) Patent Application Publication
(10) Pub. No.: US 2024/0374476 A1
(43) Pub. Date: Nov. 14, 2024

(54) METHODS OF GENERATING MOLECULAR HYDROGEN ON THE SURFACE OF THE SKIN

(71) Applicant: Azulent, LLC, Las Vegas, NV (US)

(72) Inventors: Eugene P. PITTZ, Las Vegas, NV (US); Chen CHEN, Las Vegas, NV (US)

(21) Appl. No.: 18/662,520

(22) Filed: May 13, 2024

Related U.S. Application Data
(60) Provisional application No. 63/577,659, filed on May 11, 2023.

(51) Int. Cl. A61H 99/00; A61H 23/02; A61K 8/19; A61K 8/365; A61Q 19/00; A61Q 19/08

(52) U.S. Cl. CPC A61H 99/00; A61H 23/0245; A61K 8/19; A61K 8/365; A61Q 19/00; A61Q 19/08; A61H 2201/105; A61H 2201/169

(57) ABSTRACT

A method of generating hydrogen gas on skin can include contacting a solid base metal surface with an aqueous skin care formulation to coat the solid base metal surface with the aqueous skin care formulation. A portion of the aqueous skin care formulation can be catalytically converted to molecular hydrogen (H₂) at the solid base metal surface. The skin can be massaged using the formulation-coated solid base metal surface to generate H₂ on the skin.

### CROSS REFERENCE TO RELATED APPLICATIONS

This application claims priority to U.S. Provisional Patent Application No. 63/577,659, filed on May 11, 2023, which is incorporated herein by reference.

### BACKGROUND

Molecular hydrogen (H₂), the smallest molecule in the universe, is a source of enormous energy from the sun, other stars, and other familiar processes, such as H₂ fuel for powering vehicles and rockets. Since 2008, H₂ has been studied and used as an anti-inflammatory agent for anti-aging and numerous disease states. Widespread use of H₂ for both energy and health care has been limited by difficulty in storing and maintaining H₂ at an effective concentration in storage facilities and consumer products.

The health benefits of administering molecular hydrogen to humans and animals by various routes, including IV, oral, transdermal, and inhalation have been well studied and documented. Initially, molecular hydrogen (H₂) was believed to function as an antioxidant by direct reaction with hydroxyl radicals and peroxynitrite, while leaving the signaling reactive oxygen species superoxide and hydrogen peroxide—unchanged. Molecular hydrogen (H₂) is used for wellness and, antiaging treatments, as well as prevention and treatment of numerous diseases. H₂ is readily absorbed into tissues, including the integument. The duration of H₂ in the body is short-lived, since it readily diffuses in and out of tissues. Oral dosing with H₂-water does not generate detectable H₂ diffusing through the skin. In contrast, topical application of H₂-containing skin care products allows for detecting H₂ that has reached the lower layers of the integument. Deep skin penetration, to mitochondria, is needed for H₂ to exert its most effective antioxidant and anti-inflammatory potential—that will slow down the skin aging process.

There have been major barriers to successfully manufacture and formulate stable products that will contain and sustain H₂. H₂ can permeate most materials due to being the smallest molecule in the universe. Generation of H₂, in products, has been limited by the need to add magnesium metal powder (MMP) to formulations. MMP is difficult to manufacture in formulations and transport-due to its hazardous nature. MMP powder is not stable in aqueous skin care or other aqueous based consumer products. It will spontaneously react with water—dissipating its H₂-generating potential.

### SUMMARY

The present disclosure describes methods of generating hydrogen gas on the skin of a subject. In one example, a method of generating hydrogen gas on skin can include contacting a solid base metal surface with an aqueous skin care formulation to coat the solid base metal surface with the aqueous skin care formulation. A portion of the aqueous skin care formulation can be catalytically converted to molecular hydrogen (H₂) at the solid base metal surface. The skin can then be massaged using the formulation-coated solid base metal surface to generate H₂ on and in the skin. In some examples, the solid base metal surface, as an alloy, can be composed of at least 90.0% magnesium metal or as pure metal, at least 99.995% magnesium metal. In other examples, the solid base metal surface can be composed of at least 80.0% zinc metal alloy or at least 99.99% pure zinc metal. In still other examples, the solid base metal surface can be composed of at least 90.0% iron metal alloy or at least 99.99% pure iron metal. The thickness of the solid base metal surface can be at least 0.10 mm and no more than 30.0 mm and the solid base metal surface can be in the form of a foil, a metal rod, a spatula, a cylinder, a sphere, or a combination thereof.

In some examples, the aqueous skin care formulation can have a pH lower than 6.9 and not lower than 2.0, or a pH lower than 4.0 and not lower than 2.0. The aqueous skin care formulation can be in the form of a hydrogel, cream, lotion, serum, spray, foam, or combination thereof. The method can also include adding a USP grade organic acid selected from the group consisting of: citric acid, malic acid, adipic acid, phosphoric acid, and combinations thereof to adjust a pH of the aqueous skin care formulation. Any organic acid that lowers the pH below 6.9 can be effective at hydrogen generation when used with the said base metal. In certain examples, the solid base metal surface can be placed in a container with the aqueous skin care formulation and allowed to remain there for 1 second to 1 hour to generate 100-1400 ppb H₂ in the aqueous skin care formulation on solid base metal surface. In other examples, the aqueous skin care product can be first placed on a designated site on the skin—followed by massaging that skin site with the solid base metal surface to generate H₂ on the skin surface. The solid base metal surface can be used to massage the aqueous skin care formulation into the skin for one second to 1-hour to generate H₂ on the surface of the skin as well as into the skin. The methods can also include polishing the solid base metal surface with an abrasive instrument before contacting the solid base metal surface with the aqueous skin care formulation. The abrasive instrument can be steel wool, sandpaper, or a stainless-steel brush capable of removing oxidized materials on the solid base metal surface. The solid base metal can be washed and dried after it is polished.

The present disclosure also describes massagers that can be used with an aqueous skin care formulation to generate hydrogen gas on and in the skin of the subject. In one example, an electronic or ultrasonic facial massager includes a vibrating massage head comprising a solid base metal surface composed of at least 90% magnesium or zinc or iron configured to generate H₂ on the surface of skin, as well as in the skin, when massaging an aqueous skin care formulation into the skin. The massager can also include at least one of: lights for red light therapy, lights for blue light therapy, a temperature control, and a speed control. The solid base metal surface can include a magnesium or zinc or iron foil having a thickness of 0.05 mm to 10 mm adhered to the massage head.

The present disclosure also describes kits that can be used to generate hydrogen gas on the skin of the subject. In one example, a kit includes instructions to perform a method of generating hydrogen gas on the skin of a subject and one or more of the following: a solid base metal surface spatula, an electronic or ultrasonic skin massager with a massage head comprising a solid base metal surface, an aqueous skin care formulation, an abrasive instrument for polishing the solid base metal surface, a packet containing an acid for acidification of an aqueous skin care formulation, and a pH paper for determining the pH of an aqueous skin care formulation. In some examples, the kit can include the aqueous skin care formulation and the solid base metal spatula or the massager, and the aqueous skin care formulation can have a pH from 2.0 to 6.9 such that the aqueous skin care formulation is capable of forming H₂ when contacted with the solid base metal surface.

There has thus been outlined, rather broadly, the more important features of the invention so that the detailed description thereof that follows may be better understood, and so that the present contribution to the art may be better appreciated. Other features of the present invention will become clearer from the following detailed description of the invention, taken with the accompanying drawings and claims, or may be learned by the practice of the invention.

### BRIEF DESCRIPTION OF THE DRAWINGS

FIG. 1 shows a perspective view of an example cosmetic spatula having a solid base metal surface in accordance with an example of the present technology.

FIG. 2 shows a front view of an example vibrating massager having a solid base metal surface in accordance with an example of the present technology.

FIG. 3 shows a perspective view of another example vibrating massager having a solid base metal surface in accordance with an example of the present technology.

FIGS. 4A-4B illustrate an example method of generating hydrogen gas on skin in accordance with an example of the present technology.

FIGS. 5A-5B illustrate another example method of generating hydrogen gas on skin in accordance with an example of the present technology.

FIG. 6 shows a kit in accordance with an example of the present technology.

These drawings are provided to illustrate various aspects of the invention and are not intended to be limiting of the scope in terms of dimensions, materials, configurations, arrangements or proportions unless otherwise limited by the claims.

### DETAILED DESCRIPTION

While these exemplary embodiments are described in sufficient detail to enable those skilled in the art to practice the invention, it should be understood that other embodiments may be realized and that various changes to the invention may be made without departing from the spirit and scope of the present invention. Thus, the following more detailed description of the embodiments of the present invention is not intended to limit the scope of the invention, as claimed, but is presented for purposes of illustration only and not limitation to describe the features and characteristics of the present invention, to set forth the best mode of operation of the invention, and to sufficiently enable one skilled in the art to practice the invention. Accordingly, the scope of the present invention is to be defined solely by the appended claims.

### Definitions

In describing and claiming the present invention, the following terminology will be used.

The singular forms “a,” “an,” and “the” include plural referents unless the context clearly dictates otherwise. Thus, for example, reference to “a metal” includes reference to one or more of such materials and reference to “massaging” refers to one or more of such steps.

As used herein with respect to an identified property or circumstance, “substantially” refers to a degree of deviation that is sufficiently small so as to not measurably detract from the identified property or circumstance. The exact degree of deviation allowable may in some cases depend on the specific context.

As used herein, the term “about” is used to provide flexibility and imprecision associated with a given term, metric or value. The degree of flexibility for a particular variable can be readily determined by one skilled in the art. However, unless otherwise enunciated, the term “about” generally connotes flexibility of less than 2%, and most often less than 1%, and in some cases less than 0.01%.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

As used herein, the term “at least one of” is intended to be synonymous with “one or more of.” For example, “at least one of A, B and C” and “at least one of A, B, or C” explicitly includes only A, only B, only C, or combinations of each.

Numerical data may be presented herein in a range format. It is to be understood that such range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited. For example, a numerical range of about 1 to about 4.5 should be interpreted to include not only the explicitly recited limits of 1 to about 4.5, but also to include individual numerals such as 2, 3, 4, and sub-ranges such as 1 to 3, 2 to 4, etc. The same principle applies to ranges reciting only one numerical value, such as “less than about 4.5,” which should be interpreted to include all of the above-recited values and ranges. Further, such an interpretation should apply regardless of the breadth of the range or the characteristic being described.

Any steps recited in any method or process claims may be executed in any order and are not limited to the order presented in the claims. Means-plus-function or step-plus-function limitations will only be employed where for a specific claim limitation all of the following conditions are present in that limitation: a) “means for” or “step for” is expressly recited; and b) a corresponding function is expressly recited. The structure, material or acts that support the means-plus function are expressly recited in the description herein. Accordingly, the scope of the invention should be determined solely by the appended claims and their legal equivalents, rather than by the descriptions and examples given herein.

### Example Embodiments

Molecular hydrogen (H₂), the smallest molecule in the universe, is both a source of enormous energy and a powerful antioxidant providing numerous health benefits. H₂, being subject to quantum effects, is difficult if not impossible to store, long term, in consumer packaging. Thus, stability of H₂ in products, destined for the consumer, has been lacking. The present technology involves methods for generating H₂ directly on the skin by a consumer. Thus, there is no need to stabilize, long-term, H₂—in a skin care product—to be applied to skin or scalp.

The methods described herein can include incubating a solid base metal surface (BMS) in an aqueous skin care formulation for as little time as a as a few seconds up to longer time periods to catalytically convert a portion of the skin care formulation to H₂ gas at the metal surface. The BMS can then be used to apply the H₂-containing skin care formulation to a targeted site on the skin. The BMS can be used to rub or ‘massage’ the formulation into the skin. The BMS can then be washed or wiped free of formulation. The BMS can then be returned to the same container or another container of an aqueous skin care formulation in preparation for generating an additional application. Alternatively, the BMS and the skin care formulation can be stored until its next use. This cycle has been repeated many times, such as several hundred times or more.

In another example, an aqueous skin care formulation can be applied directly to a skin surface and then the BMS can be used to massage the skin care product into the skin. The massage process can enable the BMS to contact the skin care product thereby generating H₂ on the surface of the skin. The BMS can be used repeatedly, such as hundreds of times, for this purpose.

The skin care formulations used in the present technology can be referred to as “qualified” skin care formulations or skin care products. As used herein, “qualified” means that the skin care formulation is capable of forming H₂ gas when in contact with the solid base metal surface. In some examples, qualified skin care formulations can be aqueous. When an aqueous skin care formulation contacts the solid base metal surface, hydrogen atoms from the water in the skin care formulation can be converted to H₂ gas. Additionally, in some examples, the qualified skin care formulation can have a pH in a range that allows the skin care formulation to form H₂ when contacting the solid base metal surface. In some examples, the pH can be below 6.9. The appropriate pH range can vary depending on the specific metal of the base metal surface. Additionally, the pH can be within a range that is not irritating for the skin in some examples.

The methods described herein can be performed multiple times with the same base metal surface without abrasively polishing the BMS after each use. However, in some examples it can be useful to polish the BMS periodically, such as after 20-30 uses.

In various examples, the base metal surface can be made of magnesium, zinc, or iron. A magnesium base metal surface (MBMS) can turn any skin care or hair care product, with a pH below 6.9, into an H₂-generating product on skin, scalp, and mucous membranes. A zinc surface (ZNANODE) can turn any skin care or hair care product with a pH below 4.0 into an H₂-generating product on skin, hair, and mucous membranes. An iron surface (FEANODE) can turn any skin care or hair care product with a pH below 4.1 into an H₂-generating product on skin, hair, and mucous membranes. When using MBMS or ZNANODE or FEANODE, skin and hair care products with a pH higher than able to generate H₂ (i.e., 4.0-6.9) can be turned into an H₂-generating product by addition and mixing of an acidifier, such as citric acid.

One example method allows for use of the BMS after incubation in the skin care formulation, in its container. The BMS can be in the form of a cosmetic spatula to apply the H₂-rich cream to the targeted skin site desired, where it can be massaged into the skin, with the BMS, thereby delivering H₂. Another example method includes using the BMS to generate H₂ on skin, —by massaging the cream directly into the skin. The methods described herein can increase the speed of H₂ generation on skin, increase H₂ penetration into skin and delivery of other skin benefit treatments (e.g., red light therapy for aging skin). In another example, a vibrating massager can include massage head composed of MBMS or ZNANODE or FEANODE. Thus, the vibrating massager can be used with a qualified skin care formulation as a potent generator of H₂, on skin, when massaging the qualified skin care formulation into the skin or scalp.

In some examples, the concentration of H₂ generated can be varied by changing: the surface area of the BMS; the pH of the formulation; polishing the surface area of the BMS and/or altering the time of exposure of the BMS—to the formulation. Polishing the BMS with an abrasive tool can immediately restore the BMS to its full H₂-generating potential. The BMS can be re-used at least one hundred times and in some cases many more. Long-term stability of H₂, in the skin care formulation, is not needed for the methods described herein to effectively deliver H₂ to the skin because the H₂ is immediately generated each time the BMS is exposed to the formulation.

When using the skin massage methods, skin care products can be applied to the skin by any suitable method and in any suitable form, including liquids, sprays, foams, aerosols, hydrogels, lotions, creams, and aqueous ointments. Then, the skin care formulation can be massaged into the skin with the BMS, thereby generating H₂.

In contrast to noble metals such as gold, base metals may be distinguished by oxidizing and reacting with hydrochloric acid to form molecular hydrogen. Included are magnesium, iron, aluminum, nickel, lead, tin, carbon and zinc. Copper is included but does not react with hydrochloric acid.

The US Customs and Border Protection Agency list includes copper, lead, nickel, steel, aluminum, tin, carbon, tungsten, molybdenum, tantalum, bismuth, cobalt, titanium, cadmium, zirconium, antimony, magnesium, manganese, beryllium, chromium, germanium, gallium, vanadium, hafnium, indium, indium, rhenium, thallium, and vanadium. Their alloys are also included.

Although many base metals are capable of reacting under acidic and heating conditions to oxidize and generate H₂, magnesium, zinc and iron metals are particularly suitable for the generation H₂ under the conditions of this technology. That is, BMS of magnesium, zinc and iron can all generate H₂ when exposed to skin care formulations in containers or put on skin under the pH conditions previously discussed. These metals are also safe materials to be applied to the skin.

Moisturizing skin care entails a range of practices that support skin integrity, enhance its appearance, and temporarily relieve the symptoms of dry skin. True moisturization is augmented by good nutrition, avoidance of excessive sun exposure, and appropriate use of moisturizers. Skin moisturization is a routine daily procedure, such as skin treatment for skin that feels too dry or tight. Proper moisturization involves prevention of dermatitis and prevention of skin damage due to cracking. Moisturizing skin care is at the interface of cosmetics and dermatology practices.

The FDA defines cosmetics as products intended to cleanse or beautify, for instance, makeup and lipstick. A separate category exists for medications, which are intended to diagnose, cure, mitigate, treat, or prevent disease, or to affect the structure or function of the body (e.g., sunscreens and acne products). Some products, such as moisturizing sunscreens and anti-dandruff shampoos, are regulated within both categories.

Skin ageing is associated with increased wrinkles and sagging. Fine lines, vascular bruising, dark spots, itchy, dry skin can, overtime, become more distinct. Although wrinkles occur with age, smoking and excess sun exposure can worsen the appearance of wrinkles. As humans spend time exposed to the sun, they are not aware of the long-term adverse effects. As time progresses, sunspots, chronic dryness, growing fine lines deepening wrinkles, and cancers can occur from sun exposure. The exposure to UV, in sun exposure, makes skin less ecstatic and sagging. Skin problems, including itching associated with dryness—are common in the elderly.

Skin care products claiming to be moisturizers help with reduction of the appearance of skin dryness, but less is known about their long-term, and possible adverse effects. Hydration of not only the stratum corneum but also of the epidermis, as well, can activate enzymes and epigenetic processes that can further generate the ‘natural moisturizing factor’ that keeps our skin supple and moisturized over a longer period of time. However, there may be a ‘cost’ for gaining this ‘skin benefit.’ That is, due to enhanced mitochondrial metabolism, toxic reactive oxygen species (ROS) are produced in skin-which can offset the benefits of moisturization. Some ROS may penetrate the skin due to increased moisturization. Incorporating H₂ into moisturizing skin care formulations, applied to skin, can reduce the toxic effect of ROS on sub-clinical inflammation and the concomitant aging of the integument.

In certain examples, methods of generating hydrogen gas on the skin of a subject can include placing a solid base metal surface in contact with a qualified aqueous skin care formulation that is either in a container or on the surface of the skin. The solid base metal surface will generate molecular hydrogen (H₂) in the layers of the formulation closest to the solid base metal surface. The consumer can then use the formulation-coated base metal surface to massage the skin care formulation into the skin-thereby generating H₂ on the surface of the skin.

In some cases, the purity of the base metal surface can affect the generation of H₂ gas. In certain examples, the solid base metal surface can be composed of at least 90.0% and preferably 99.995% by weight of magnesium metal. In other examples, the solid base metal surface can be composed of at least 80.0% and preferably 99.99% by weight of zinc metal. In still other examples, the solid base metal surface can be composed of at least 90.0% and preferably 99.99% by weight of iron metal.

The surface area of the solid base metal surface can be at least 0.15 sq. cm. and as high as 3,500 sq. cm in some examples. In further examples, the thickness of the solid base metal surface can be at least 0.10 mm and no thicker than 30.0 mm. The base metal surface can be in the form of a foil, metal rod, a spatula, a cylinder, a sphere, or any solid form capable of massaging the skin.

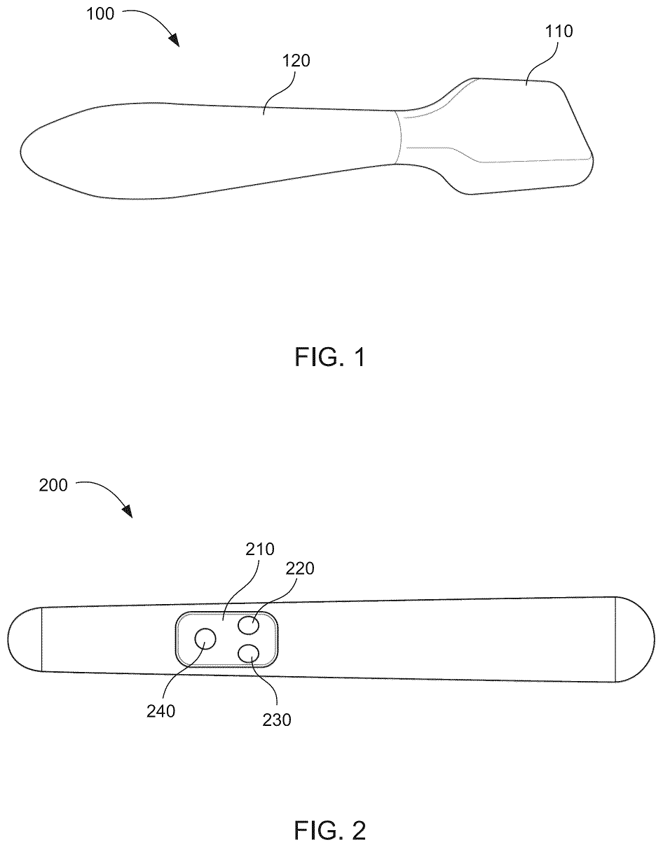

One example solid base metal surface is illustrated in FIG. 1. This figure shows a perspective view of a base metal cosmetic spatula 100. This cosmetic spatula can be used to spread a qualified skin care formulation on the skin and massage the formulation into the skin. In some examples, the entire spatula can be formed from a base metal. As explained above, the base metal can be magnesium, zinc, or iron. In further examples, the cosmetic spatula can have a core formed of another material and a surface formed of the base metal, such as by coating the surface in the base metal, plating the surface in the base metal, applying a foil of the base metal to the surface, and so on. In other examples, a portion of the surface of the cosmetic spatula can be made from the base metal and another portion of the surface can be another material. For example, a head 110 of the cosmetic spatula can be made of base metal and a handle 120 of the cosmetic spatula can be made from another material. The portion of the surface made from the base metal can be the portion used to contact the skin care formulation and the skin of the subject. In this example, the “solid base metal surface” can refer to any surface of the cosmetic spatula that is made from the base metal.

Another example solid base metal surface is illustrated in FIG. 2. This figure shows a front view of a vibrating massager 200. This example vibrating massager has a cylindrical or wand shape. In some examples, the entire exterior surface of the massager can be made of the base metal, or the entire surface except for a control panel 210 can be made from the base metal, or a portion of the surface can be made from the base metal. Thus, the “solid base metal surface” can refer to whatever portion of the surface of the massager is made from the base metal. In this example, the vibrating massager includes a control panel that can include a vibration control 220 and optionally other controls such as a temperature control 230 or speed control 240.

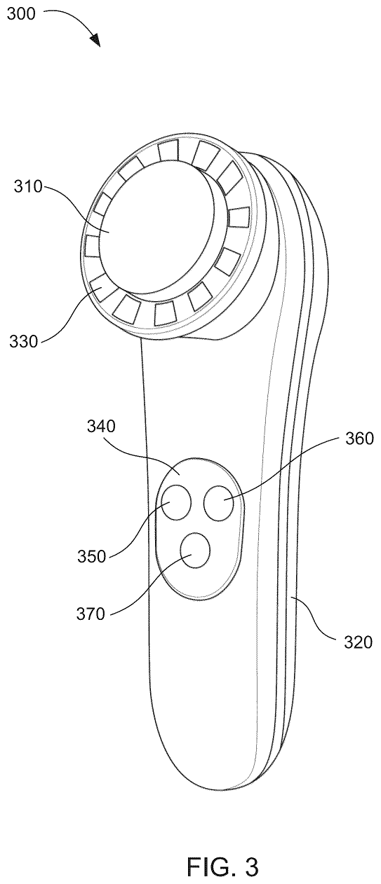

Another example solid base metal surface is illustrated in FIG. 3. This figure shows a perspective view of another example vibrating massager 300. This example includes a vibrating massage head 310 on a grip body 320. The vibrating massage head can be made of the base metal. Therefore, the solid base metal surface can refer to the base metal massage head in this example. This massager also includes additional features, including light emitting diodes (LEDs) 330 for light therapy surrounding the massage head. The LEDs can be used for red light therapy, blue light therapy, a combination thereof, or other light therapy. The massager also includes a control panel 340 that includes a speed control 350, a temperature control 360, and a light therapy control 370. Other controls can also be included in further examples.

In some examples, the qualified skin care formulation can be an aqueous acidic formulation can be a skin care product having a pH lower than 6.9 and not lower than 2.0. In further examples, the acidic formulation can be a skin care product having a pH lower than 4.0 and not lower than 2.0. In various examples, the skin care product can be in the form of a hydrogel, cream, lotion, serum, spray, or any other suitable form of formulation used by the skin care consumer.

An acidifying agent can be added to the skin care formulation to adjust the pH of the skin care formulation. Any USP grade organic acid, and combinations of such, such as citric acid, malic acid, adipic acid or phosphoric acid, can be added to the aqueous skin care formulation to achieve a pH of between 2.0 and 6.9 or between 2.0 and 4.0 or between 2.0 and 4.1.

In some examples, the aqueous skin care formulation further comprises a nitrite salt such that the massaging the skin will also generate nitric oxide in the skin. Non-limiting examples of suitable nitrite salts can include nitrite-containing foods, amyl nitrite, and mineral nitrite salts such as, but not limited to, nitrite salts of potassium, sodium, magnesium, calcium, ammonium and lithium. As a general guideline, the nitrite salt can be present at 0.01 to 25% by weight of the formulation.

In some examples, a method of generating hydrogen gas on the skin can include placing the solid base metal surface in a container with the aqueous skin care formulation. The solid base metal surface can be allowed to remain in the container in contact with the aqueous skin care formulation for a time period to generate H₂ on the base metal surface. In some examples, the time period can be 1-5 seconds and the concentration of H₂ generated can be 100-500 ppb H₂ in the formulation on the surface of the base metal surface. In other examples, the time period can be 5-30 seconds and the concentration H₂ generated can be 400-900 ppb H₂ in the formulation on the surface of the base metal surface. In further examples, the time period can be 0.5-60 minutes and the concentration of H₂ generated can be 500-1400 ppb H₂ in the formulation on the surface of the surface of the base metal. In still further examples, the time period can be 2 hours to two years and the concentration of H₂ generated can be 800-1400 ppb H₂ in the formulation on the surface of the base metal.

In some examples, the skin care formulation can be in a container with a cover, and the container can be made of glass or plastic or metal or any material compatible with the said formulation ingredients. The solid base metal surface can be placed in the container, at least partially, to contact the solid base metal surface with the skin care formulation. The solid base metal surface, now coated with H₂-containing skin care formulation, can then be removed, by the user, from the container and contacted with the skin (which can include mucus membranes) of an individual (either the user or another person being treated by the user) for the purpose of administering the formulation to the individual's skin or mucous membranes.

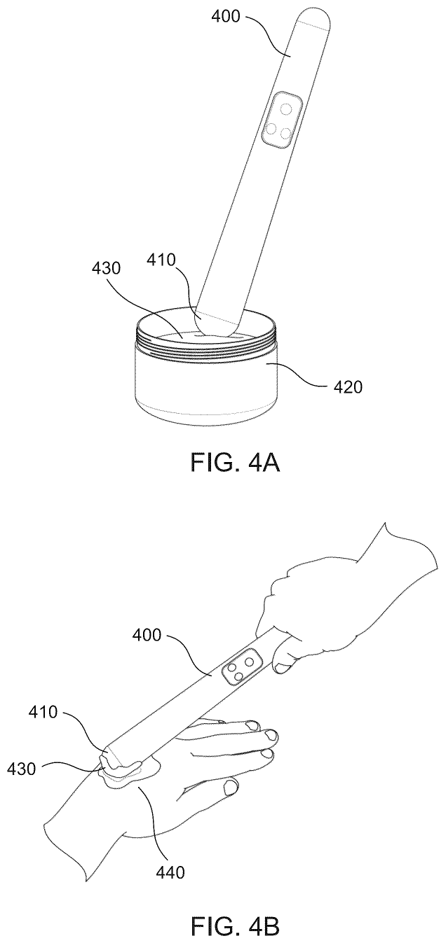

FIG. 4A shows an example of a vibrating massager 400 having a solid base metal surface 410 that is placed into a container 420 of an aqueous skin care formulation 430. The massager is dipped in the formulation so that the solid base metal surface contacts the formulation. The massager can be left in the container for a period of time, during hydrogen gas begins to form at the solid base metal surface. FIG. 4B shows a following step in which the massager is removed from the container and then is used to massage skin 440 of the user. The solid base metal surface is still coated with the skin care formulation, so that the skin care formulation is applied to the skin simultaneously with massaging the skin of the user. The hydrogen gas that had formed in the skin care formulation is thus applied to the skin. The solid metal surface of the massager can also continue to generate additional hydrogen gas while massaging the skin, so that additional hydrogen gas is applied to the skin.

In other examples, the method of generating hydrogen gas can include placing an aqueous skin care formulation on a designated site on the skin first, and then massaging the skin sit with the solid base metal surface. In this example, the solid base metal surface is not dipped in the skin care formulation before massaging the skin with the solid base metal surface. In certain examples, the solid base metal surface can be used to massage the skin care formulation into the skin for a time period from 1 second to 1 hour to generate H₂ on the surface of the skin. The skin care formulation used in these methods can include any suitable skin care product or hair care product, such as a serum, lotion, cream, spray, gel, or foam delivered from any suitable container.

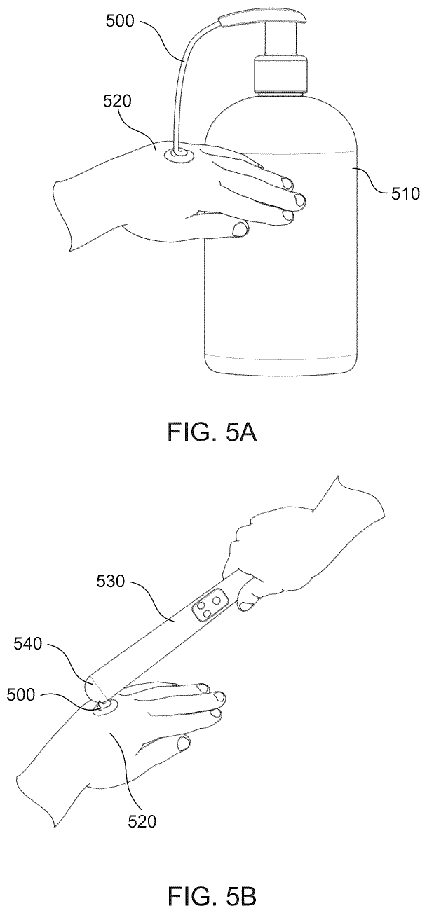

FIGS. 5A and 5B illustrate an example method in which the skin care formulation is first applied to the skin. FIG. 5A shows a skin care formulation 500 being applied from a container 510 to the skin 520 of a user. In this example, the container is a pump bottle and the skin care formulation is a lotion. FIG. 5B shows a vibrating massager 530 having a solid base metal surface 540 that is being used to massage the skin care formulation into the skin at the designated site where the skin care formulation was applied. The skin care formulation can further be spread onto other areas of the skin using the massager. While the massager is used to massage the skin, hydrogen gas is simultaneously generated at the solid base metal surface. The hydrogen gas can diffuse into the skin to provide therapeutic effects.

After using a solid base metal surface to generate hydrogen gas in the methods described herein, the solid base metal surface can be washed and/or wiped clean for additional use, for the same purpose. In some examples, the methods can include polishing the base metal surface with an abrasive instrument before it is placed in a container of skin care formulation or before massaging the formulation into skin. The abrasive instrument can be fine steel wool, fine sandpaper, a stainless-steel brush, or any other abrasive instrument that can remove oxidized materials on the surface of the used base metal surface. The base metal surface can be re-used to generate and/or transfer H₂ to the skin of the consumer. In some examples, the base metal surface can be re-used at least one hundred times and as many as 1,000 times.

In some examples, the solid base metal surface can be on a vibrating massage head of an electronic or ultrasonic facial massager. The electronic massager can have other features including red and blue light therapy, vibrating massage head, temperature control and speed control. In certain examples, the solid base metal surface can be made by retrofitting an electronic or ultrasonic facial massager by adhering a magnesium or zinc or iron foil that has a thickness of 0.05 mm to 10 mm to the massage head. In certain examples, a magnesium or zinc or iron foil can be adhered to the massage head with any waterproof glue.

The methods described herein can also be performed using a kit. The kit can include a solid base metal surface that can be in any of the forms described herein. The kit can also include instructions for use of the kit. Kits can also include one or more of the following: base metal surface spatulas; an electronic or ultrasonic skin massager with a massage head of an effective base metal surface (BMS); a proprietary skin care formulation designed for use with a specific BMS; a skin care formulation that was not specifically designed for use with a BMS; an abrasive instrument for polishing the base metal surface; a packet for acidification of a high pH product; pH paper for determining the pH of a product to be used.

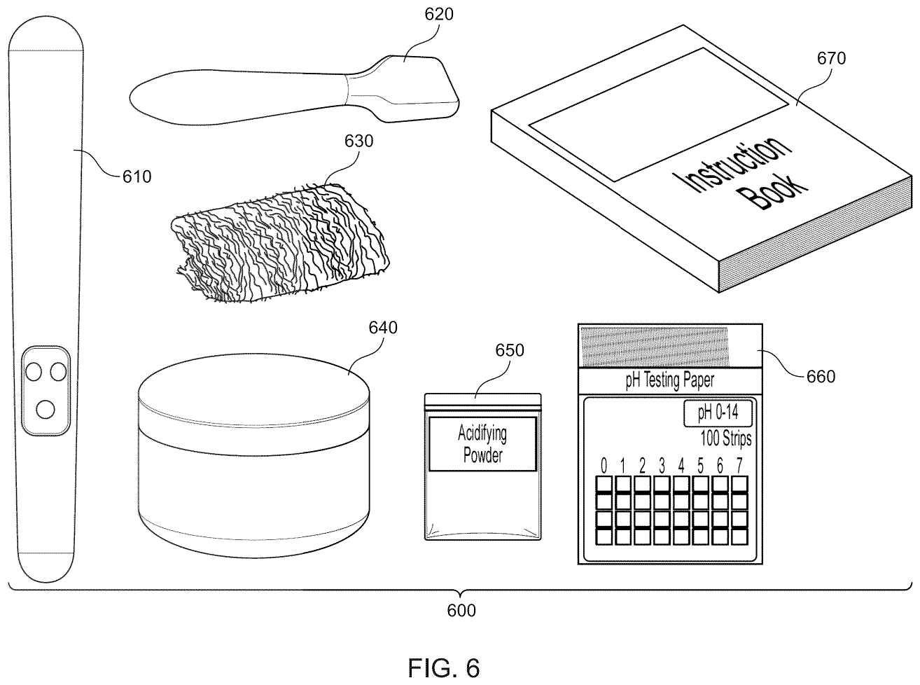

FIG. 6 illustrates an example kit 600. This kit includes a vibrating massager 610 with a solid base metal surface, a solid base metal cosmetic spatula 620, steel wool 630 for polishing the base metal surfaces, a container of skin care formulation 640, a packet of acidifying agent 650 to adjust the pH of the skin care formulation, pH test paper 660 for determining the pH of the skin care formulation, and a set of instructions 670 to instruct a user in the use of the kit. In some examples, these items or a combination of one or more of the items can be packaged together.

In further examples, a skin care formulation can be specifically marketed or developed to be capable of use with a magnesium metal surface to generate H₂. In certain examples, the pH of the skin care formulation can be adjusted to be in the range of 2.0 to 6.5. In further examples, a skin care formulation can be specifically marketed or developed to be capable of use with a zinc metal surface to generate H₂. In certain examples, the pH of the skin care formulation can be adjusted to be in the range of 2.0 to 4.0. In still further examples, a skin care formulation can be specifically marketed or developed to be capable of use with an iron metal surface to generate H₂. In certain examples, the pH of the skin care formulation can be adjusted to be in the range of 2.0 to 4.1.

### EXAMPLES

### Example 1: Generation of Molecular Hydrogen (H₂) in a Leading Moisturizing Cream—by Use of a Magnesium Base Metal Surface (MBMS)

### Background

H₂ is known to be generated from base metals when they are immersed in aqueous hydrochloric acid and other acids. Studies were conducted to determine if H₂ can be generated in a leading skin moisturizing cream by use of an MBMS and, if so, how that could benefit from application of the H₂-activated cream to the skin.

### Materials and Methods

CeraVe® Moisturizing Cream was purchased on Amazon. The MBMS (16 mm×90 mm; 493.6 sq. cm was purchased ‘online’ from a Chinese source (G378-1PCMAGNE).

The pH measurement was taken with a calibrated (pH 4 and 7) Extech ExStik® PH100. The pH was 5.1. The molecular hydrogen (H₂) was measured with a Trustlex ENH-1000 H₂ Meter adapted for skin surface measurements.

### Procedures and Results

After abrasively polishing both the sensor on the Trustlex and the entire surface of the MBMS, three-fourths of the length of the MBMS was dipped into a 539-gram jar (85 mm high) of CeraVe® Moisturizing Cream. The MBMS was allowed to ‘stand’ in the Cream for 30-seconds. The 3-grams of Cream that adhered to the MBMS was then transferred to the right, lower forearm of the adult male subject. MBMS was used to ‘massage’ the Cream into the skin for 30 seconds. Thereafter, the H₂ on the skin surface was found to be 420 ppb.

### Discussion

This Study demonstrates the feasibility of using the MBMS to both generate H₂ ‘in situ’ in the Cream and to transfer H₂-containing cream to the integument.

### Example 2: MBMS Affects Generation of Molecular Hydrogen (H₂) on the Surface of the Skin

### Background

Given that it was unexpectedly discovered that MBMS can generate H₂, in CeraVe® Moisturizing Cream, and that it can be used to transfer an H₂-containing cream to the skin, as well as massaging H₂-containing cream into skin, it was decided to do further investigation of an alternate procedure by which the Cream is first put onto a site on the skin and then it is massaged into the skin with the MBMS.

### Materials and Methods

CeraVe® Moisturizing Cream was purchased on Amazon. The MBMS (16 mm×90 mm; 493.6 sq. cm was purchased ‘online’ from China (G378-1PCMAGNE).

The pH measurement was taken with a calibrated (pH 4 and 7) Extech ExStik® PH100. Molecular hydrogen (H₂) was measured with a Trustlex ENH-1000 H₂ Meter adapted for skin surface measurements.

### Procedures and Results

Two grams of unadulterated CeraVe® Moisturizing Cream was removed from its jar and placed on the inner aspect of the forearm with a wooden tongue depressor. The MBMS, described in Example 1, was used to massage the Cream into the skin site—for 30 seconds. The concentration of H₂ on the skin surface was then measured with the Trustlex H₂ Meter. The maximum H₂ level was found to be 540 ppb.

### Discussion

This Example demonstrates that by massaging unadulterated CeraVe® M C into the skin, with the MBMS, a considerable amount of H₂ is generated. That this H₂ is bioavailable will be demonstrated—as shown in Example 4—below. It can be hypothesized that the MBMS can be used to generate H₂ on the surface of the skin by massaging any skin care product, with a pH lower than 6.9, into the skin.

### Example 3: Testing a Popular Lotion Delivered from a Pump Bottle

### Background

In Example 2, it was demonstrated that applying a Cream to the skin and massaging that cream into the skin with the MBSB will generate a considerable amount of H₂ on the surface of the skin. This study determines the amount of H₂ generated by first pumping the lotion onto the skin and then by massaging lotion into skin with the MBMS.

### Procedures and Results

1.8-grams of Vaseline Intensive Care® Advanced Repair Lotion (pH 5.77) was pumped from its bottle onto the inner aspect of the forearm. The MBMS, described in Example 1, was used to massage the Lotion into the skin site—for 30 seconds. The concentration of H₂ on the skin surface was then measured with the Trustlex H₂ Meter. The H₂ level was maximum at 1047 ppb.

### Discussion

The results of this Study show that application of a lotion to skin before massaging that site, with the MBMS, may be more effective at generating H₂ than application of a cream. Massaging a lotion offers more contact of the MBMS with a lotion content than does a thixotropic cream. Regardless, the results support the notion that applying any topical dosage form and massaging it into the skin, with the MBMS-will generate a significant quantity of H₂ on the surface of the skin.

### Example 4: Bioavailability of Molecular Hydrogen (H₂) Delivered Transdermally from an H₂-Infused Moisturizing Cream

### Introduction

In the previous Examples, it has been demonstrated that messaging H₂-infused CeraVe MC into skin can generate substantial skin surface H₂. Here, it is demonstrated that topical application of an H₂ CeraVe formula can deliver H₂ to the lower layers of the skin.

### Material and Methods

CeraVe® Moisturizing Cream was purchased “online” from Amazon. A single dose packet of a powder formula containing zinc metal powder was added to 40 mL of CeraVe MC, in a 100 mL glass container and mixed with a utensil for 60 seconds. The mixture was allowed to ‘stand’ until use. Trustlex H₂ measurement determined that the formulation generated close to 1.1 ppm H₂. Before application of the H₂-Cera Ve MC to the skin, baseline breath H₂ measurements were taken with a Forensics Detectors modified F-600 H₂ Detector for breath H₂ measurements. Application consisted of smearing generous portions of the H₂-infused CeraVe MC over the arms, legs, and chest. Measurements of breath H₂ took place over the time course as shown below.

### Results

The breath test results for application of H₂ CeraVe MC to the body are shown in Table 1 below.

TABLE 1

| | H₂-CeraVe Transdermal Results | | |
|---|---|---|---|
| | Time | Breath | % Change |
| Activity | Minutes | H₂, ppm | from Baseline |
| baseline | 0 | 6.9 | n/a |
| measure | 10 | 12.1 | 75.4 |
| measure | 20 | 12.6 | 82.6 |
| measure | 35 | 9.1 | 31.9 |
| measure | 55 | 9.1 | 31.9 |
| measure | 110 | 5.9 | −14 |

### Discussion

The results presented in Table 1 indicate:
(1) Topical application of H2 CeraVe® MC can increase breath H2.
(2) Topical application can affect skin penetration to the low layers of the skin.

### Example 5: H₂ Generation in CeraVe® Moisturizing Cream—as a Function of Exposure Time to an MBMS

### Background

In Example 1, it has been demonstrated that an MBMS, when incubated with a qualified skin care formulation, can transfer an H₂-containing formulation to the skin. This study involves:
(1) The effect of exposure time of MBMS to a skin care formulation-on transfer of the H₂-infused formulation to a surrogate for skin;
(2) the effect of stirring the formulation vs. just dipping the MBMS, into skin-on transfer of an H₂-infused formulation to a surrogate for skin.

### Materials and Methods

CeraVe® Moisturizing Cream was purchased on Amazon. The MBMS (14 sq. cm) was purchased ‘online’ from China (XUO1Y11X97). The pH measurement was taken with a calibrated (pH 4 and 7) Extech ExStik® PH100. Molecular hydrogen (H₂) was measured with a Trustlex ENH-1000 H₂ Meter adapted for surface measurements.

A wooden tongue depressor was used as a surrogate for skin.

### Procedures and Results

For Study (1), the MBMS was immersed in CeraVe® Moisturizing Cream—for various periods of time. The MBMS, with cream adhered to it, was removed from its jar and the cream on the MSMB was rubbed, for 30-seconds, onto a wooden tongue depressor. The Cream on the tongue depressor surface was then evaluated for H₂—using the Trustlex H₂ Meter.

For Study (2), the MBMS was immersed in Cera Ve® Moisturizing Cream—for one minute. For Protocol (A), the MBMS was left in the cream for 1-minute, undisturbed. For Protocol (B), the MBMS was used to stir the cream for 1-minute. For both Protocols, the MBMS, with cream adhered to it, was removed from its jar and the cream on the MBMS was rubbed for 30-seconds onto a wooden tongue depressor. The cream on the tongue depressor surface was then evaluated for H₂ using the Trustlex H₂ Meter.

TABLE 2
Study 1 Results
H₂ Generation on Tongue Repressor Due to
Incubation of MBMS with CeraVe ® Moisturizing Cream

| Seconds | H₂, ppb |
|---|---|
| Immersion | On Stick |
| 10 | 250 |
| 20 | 325 |
| 30 | 342 |
| 50 | 398 |
| 120 | 655 |
| 240 | 793 |

### Discussion

The results show a trend for increased H₂ on the tongue depressor—with time of exposure of the MBMS to the Cera Ve® Moisturizing Cream. Certainly, the consumer would prefer a lower exposure time. One means of increasing H₂ generation while reducing exposure time is to provide more surface area to contact Cera Ve® MC. For example, if a 20-second exposure of an MBMS, with 14 sq. cm. surface area, generates 325 ppm, an MBMS with 42 sq. cm. of surface area would be expected to generate the equivalent of close to 975 ppb. H₂.

TABLE 3
Study 2 Results

| H₂, ppb | H₂, ppb | |
|---|---|---|
| Static | Mixing | % H₂ Loss |
| 1- Minute | 1- Minute | By Mixing |
| 684 | 480 | −30 |
| 643 | 328 | −49 |
| 571 | 640 | +12 |
| 620 | 437 | −30 |
| 630 | 471 | −25% |

### Discussion

The results show that mixing Cera Ve® Moisturizing Cream with a MBMS reduces the transfer of H₂ to the skin surrogate. Therefore, it is preferred to dip the MBMS into the CeraVe M C and leave it there undisturbed. Actually, when thought out, more H₂ should be present on the undisturbed MBMS-since mixing can ‘shear’ layers of H₂-rich cream off of the MBMS before transfer of the Cream to the skin surrogate.

### Example 6: Generation of H₂ in Acidified and Unadulterated CeraVe® Moisturizing Lotion by Various BMS

### Background

This Example addresses the following questions: Are magnesium base metal sheets (MBMS) unique in generating H₂ in skin care products? Do any other base metal sheets generate H₂-in skin care products? What is the effect of acidification of Cera Ve® M C with 2% citric acid, on H₂ generation? Can metals that are common contaminants of magnesium metal leach out into CeraVe® MC from a BMS source of such metal?

### Materials and Methods

The 2% w/w citric acid CeraVe® Moisturizing Cream was prepared by addition of 6-grams of anhydrous citric acid to 294 grams of CeraVe M C—and mixing for 5-minutes.

The 40 sq. cm anodes (36 sq. cm. immersion area) were obtained ‘online’ from China.

Molecular hydrogen (H₂) was measured with a Trustlex ENH-1000 H₂ Meter adapted for surface measurements. BMS weights were measured with an analytical balance sensitive to 0.1 mg.

### Procedures and Results

After taking the initial weight of the base metal anodes listed in Table 4, below, a base metal anode was immersed in CeraVe® Moisturizing Cream or in 2% w/w citric acid-CeraVe® Moisturizing Cream—for 10-minutes. After removal of the coated base metal anode, which was coated with Cream, the Cream was transferred to tongue depressor. After polishing the sensor on the Trustlex, it was used to measure the H₂ concentration on the wooden tongue depressor. The results are presented in Table 4, below.

TABLE 4

| | Immersion in 2% | | | | |
|---|---|---|---|---|---|
| | Citric Acid - | | | | |
| | CeraVe ® MC | | Immersion on CeraVe ® MC | | |
| | | Base Metal | | Base Metal | |
| Base | | Wt. Change | | Wt. Change | Surface Area |
| Metal | H₂, PPM | Mg. | H₂, PPM | Mg. | (sq. Cm). |
| Mg 1 | 540 | 0.9 | 601 | 0.2 | 400 |
| Mg 2 | 220 | 1.6 | 607 | 1.2 | 14 |
| Cu | 0 | 0.8 | 0 | 0.4 | 36 |
| Zn | 611 | 0.1 | 0 | 0.5 | 36 |
| Al | 0 | −0.4 | 0 | 0.7 | 36 |
| Fe | 493 | −3.6 | 0 | 0.0 | 36 |
| Ni | 0 | 1.3 | 0 | 0.3 | 36 |
| Sn | 0 | 0.6 | 0 | 1.0 | 36 |
| C | 0 | 1.3 | 0 | 2.8 | 36 |
| Pb | 0 | −1.3 | 0 | 0.5 | 36 |

### Discussion

The results presented in Table 4, show that the magnesium base metal sheet (MBMS) generates H₂ due to incubation with Cera Ve MC and 2% w/w Citric Acid-CeraVe® MC. A magnesium metal anode with a smaller surface area (14 vs. 400 sq. cm.) transfers a similar ppb H₂. The larger anode, of course, will transfer a larger quantity of H₂-Cream.

The results presented in Table 4 also show that none of the other metals evaluated generates H₂—from unadulterated Cera Ve® M C—which has a pH of around 5.1. However, when immersed in 2% Citric Acid-CeraVe® MC, both the ZNANODE and the FEANODE generated significant H₂. In Example 13 and 14, below, it will be shown that a zinc base metal sheet (ZNANODE) and the iron base metal sheet (FEANODE) can generate H₂—when the contact formulation has a pH below 4.0-4.1.

The base metals evaluated, here, are potential contaminants of an MBMS. For immersion in unadulterated CeraVe® MC, none of the base metal sheets lost weight. Aluminum (Al), iron (Fe) and lead (Pb) sheets had significant loss of weight when undergoing a 10-minute immersion in 2% Citric Acid—CeraVe M C. Since these metals could prove to be contaminants of an MBMS, testing for these metals in skin care formulations and surrogates of skin-exposed to MBMS is warranted.

### Example 7: Generation of H₂ and Nitric Oxide on an N1O1 Product Treated Skin Surface—by Massaging the Treated Site with an MBMS

### Background

N1O1 is a skin care product in that it separates its ingredients into two compartments—for stability purposes. Mixing a dose of ingredients, from both compartments—after application to skin, can produce mild skin erythema-due to the topical generation of nitric oxide (NO). Nitrite and ascorbic acid in an acidic environment are required for this NO generation. The ascorbic acid and the nitrite are contained in separate compartments—until it is desired for the NO—production takes place—by mixing aliquots of the two separate compartments.

This Study was conducted to determine if mixing and massaging N1O1 into skin with a MBMS will generate molecular hydrogen and NO on the surface of the skin.

### Materials and Methods

N1O1 was obtained from Pneuma Nitric Oxide, LLC (Austin, TX). The MBMS (16 mm×90 mm; 493.6 sq. cm was purchased ‘online’ from a Chinese source. (G378-1PCMAGNE). The pH measurement was taken with a calibrated (pH 4 and 7) Extech ExStik® PH100. Molecular hydrogen (H₂) was measured with a Trustlex ENH-1000 H₂ Meter adapted for skin surface measurements.

### Procedures & Results

Both the surfaces of the Trustlex sensor and the MBMS were polished at the start of the Experiment. Two doses of N1O1 (0.45 g.) were deposited on a 2×2″ site of the inner aspect of the left forearm. A MBMS was used to ‘massage’ the N1O1 into the skin—for 30-seconds. Slight erythema was seen to develop against a yellow background. The H₂ on the skin surface was found to be maximal at 267 ppb. The skin surface pH was found to be high at 9.49.

A control experiment was conducted where two doses of N1O1 (0.50 g.) were deposited on a 2×2″ site of the inner aspect of the right forearm. The subject's fingers were used to ‘massage’ the N1O1 into the skin—for 30-seconds. Slight erythema was seen to develop against a yellow background. The H₂ on the skin surface was found to be zero ppb. The skin surface pH was found to be 5.40.

### Discussion

The results demonstrate the feasibility of massaging N1O1 treated skin, with a MBMS in generation of both H₂ and NO on the surface of the skin. Of interest is the observation that by massaging N1O1 into the skin, the pH rises from 5.40 (control) to 9.49. This phenomenon is indicative of the generation of magnesium hydroxide, or another base on the skin surface-due to massaging N1O1 with MBMS into skin.

Both NO, by generation of erythema, and H₂, by transdermal studies (Example 4), have been shown to penetrate to the lowest layers of the skin. Both NO and H₂ have been associated with slowing aging. Therefore, a product capable of delivering both H₂ and NO to skin is expected to demonstrate promising results on reducing and/or slowing the progression of markers of aging skin.

### Example 8: Repetitive Testing of 1% Citric Acid in CeraVe® MC for—Generation and Transfer of Molecular Hydrogen (H₂) Due to Use of MBMS

### Background

It is possible to modify commercial skin care products in order to increase the generation of H₂ due to interaction with MBMS. Thus, a study was conducted to determine the effect of lowering the pH of CeraVe® MC on the generation and transfer of H₂ by MBMS. Here, measurements are taken of the H₂ generated, multiple times, when the pH of CeraVe® M C is lowered from 5.0 to 2.8.

### Methods and Materials

CeraVe® Moisturizing Cream was purchased on Amazon. The MBMS (14-sq. cm) was purchased ‘online’ from China. The pH measurement was taken with a calibrated (pH 4 and 7) Extech ExStik® PH100. Molecular hydrogen (H₂) was measured with a Trustlex ENH-1000 H₂ Meter adapted for skin surface measurements. A 1% anhydrous citric acid (Kraft Chemical Co.) formulation was prepared by mixing three grams of citric acid with 297 grams of CeraVe Moisturizing Cream. It was stored in a 500-mL covered glass jar. It was used in the tests described in Table 5-below. The baseline H₂ content of the Cera Ve® MC was 0 ppb, and the pH of the acidified Cream was 2.8.

### Procedures

Before each test, the following procedures were conducted: The weight of the Cream in the jar was determined; The MBMS was polished with an adhesive cloth, rinsed, and dried; The weight of the MBMS was measured on an analytical balance; The MBMS was placed in the Cream, agitated for 10-seconds, and left to incubate—for 5-minutes; The Cream adhering to the MBMS was transferred to a tongue depressor; The sensor on the Trustlex was polished-before use; The highest reading of H₂ on the Trustlex was recorded-after 15-seconds of observing readings; The weight of the Cream remaining in the jar was recorded and the amount of Cream removed from the jar, calculated; After removing the Cream from the MBMS, its weight was measured and the weight change of the MBMS, determined; The pH of the Cream on the ‘stick’ was measured.

### Results

The results are presented in Table 5.

TABLE 5

Change in

| | | | MBMS | Cream |
|---|---|---|---|---|
| Test # | H₂, ppb | SpH | Wt. (mg.) | Transfer (g.) |
| 1 | 607 | 2.78 | 0.0 | 1.2 |
| 2 | 525 | 2.77 | −1.6 | 1.0 |
| 3 | 831 | 3.00 | −0.5 | 0.7 |
| 4 | 548 | 2.71 | −0.5 | 1.1 |
| 5 | 677 | 2.81 | −0.5 | 1.3 |
| 6 | 347 | 2.79 | 0.6 | 1.0 |
| 7 | 247 | 2.81 | −1.7 | 1.2 |
| 8 | 676 | 2.76 | −1.5 | 1.0 |
| 9 | 408 | 2.78 | −0.3 | 0.9 |
| 10 | 549 | 2.79 | −0.4 | 2.1 |
| 11 | 599 | 2.81 | −0.6 | 3.9 |
| 12 | 721 | 2.84 | −0.7 | 0.7 |
| 13 | 522 | 2.74 | −1.1 | 1.5 |
| 14 | 731 | 2.81 | 1.0 | 0.9 |
| 15 | 750 | 2.92 | −2.6 | 1.2 |
| 16 | 714 | 2.82 | 0.5 | 1.0 |
| 17 | 712 | 2.73 | −0.3 | 1.9 |
| 18 | 728 | 2.86 | −0.8 | 0.8 |
| 19 | 731 | 2.85 | −0.6 | 0.7 |
| 20 | 712 | 2.97 | −0.3 | 1.2 |
| 21 | 745 | 2.92 | −1.0 | 1.2 |
| 22 | 652 | 2.97 | −0.6 | 1.6 |
| 23 | 746 | 2.88 | −0.6 | 0.9 |
| 24 | 754 | 2.80 | −0.5 | 0.9 |
| 25 | 718 | 2.89 | −0.1 | 0.7 |
| 26 | 741 | 3.03 | −1.0 | 1.0 |
| 27 | 675 | 2.99 | −0.3 | 0.9 |
| 28 | 724 | 2.71 | −0.2 | 1.2 |
| 29 | 654 | 2.9 | −0.8 | 1.1 |
| 30 | 716 | 2.76 | −0.3 | 1.1 |
| Av. | 649 | 2.84 | −0.6 | 1.2 |

SpH—Surface pH

### Discussion

The results demonstrate that with polishing of the MBMS, it can be used to transfer and generate an H₂-rich cream on the surface of a skin surrogate, e.g., a wooden tongue depressor—for at least 30-uses. The 1.2-gram average delivery of cream from the jar due the removal of the MBMS is sufficient quantity for treatment of an area the size of the face but is of a low value for an application to large areas of the skin. This shortfall is easily remedied—by increasing the surface area of the MBMS—to be used. The loss of weight from the MBMS is due to both polishing and erosion of the MBMS into the cream. Additional studies will be conducted to determine the amount of loss due to erosion into the Cream.

### Example 9: Repetitive Testing of 1% Citric Acid in L′Oréal's REVITALIFT® Triple Power Anti-Aging Lotion—for Generation and Transfer of Molecular Hydrogen (H₂) Due to MBMS

### Background

Example 9, above, provided evidence that a 1% citric acid—when formulated with CeraVe® M C repetitively generates H₂ over the course of 30-tests, when the MBMS is used to generate the H₂ on a surrogate—for skin. Here, the results are shown of testing L′Oréal REVITALIFT® Triple Power Anti-Aging Lotion for generation of H₂ using MBMS for H₂ activation.

### Methods and Materials

L′Oréal REVITALIFT® Triple Power Anti-Aging Lotion was purchased on Amazon. The MBMS (14-sq. mm) was purchased ‘online’ from China.

The pH measurement was taken with a calibrated (pH 4 and 7) Extech ExStik® PH100. Molecular hydrogen (H₂) was measured with a Trustlex ENH-1000 H₂ Meter adapted for skin surface measurements.

A 1% anhydrous citric acid (Kraft Chemical Co.) formulation was prepared by mixing 0.3 grams of citric acid with 29.7 grams of L′Oréal REVITALIFT® Triple Power Anti-Aging Lotion. It was stored in a 500-mL glass jar. It was used in the tests described in Table 5—below. The baseline H₂ content of L′Oréal REVITALIFT® Triple Power Anti-Aging Lotion was 0 ppb and the pH of the acidified Lotion was 4.14.

### Procedures

Before each test, the following procedures were conducted: The weight of the Lotion in the jar was determined; The MBMS was polished with an adhesive cloth, rinsed, and dried; The weight of the MBMS was measured on an analytical balance; The MBMS was immersed in the Lotion, agitated for 10-seconds, and left to incubate—for 5-minutes; The Lotion adhering to the MBMS was transferred to a tongue depressor; The sensor on the Trustlex was polished before use; The highest reading of H₂ on the Trustlex was recorded—after 15-seconds of observing readings; The weight of the Lotion remaining in the jar was recorded and the amount of Lotion removed from the jar—calculated; After removing the Lotion from the REVITALIFT® Triple Power Anti-Aging Lotion, its weight was measured and the weight change of the MBMS determined; The pH of the Lotion on the tongue depressor—was measured.

### Results

The results are presented in Table 6.

TABLE 6

Change in

| | | | MBMS | Cream |
|---|---|---|---|---|
| Test # | H₂, ppb | SpH* | Wt. (mg.) | Transfer (g.) |
| 1 | 643 | 4.14 | −0.6 | 0.6 |
| 2 | 343 | 4.14 | −1.1 | 0.5 |
| 3 | 742 | 4.05 | −0.6 | 0.7 |
| 4 | 628 | 3.98 | −0.5 | 0.7 |
| 5 | 230 | 4.18 | −0.7 | 0.4 |
| 6 | 860 | 4.12 | −1.3 | 0.7 |
| 7 | 857 | 4.07 | −0.4 | 0.4 |
| 8 | 317 | 4.07 | −0.1 | 0.6 |
| 9 | 443 | 4.28 | −2.1 | 0.6 |
| 10 | 423 | 4.23 | −0.5 | 0.5 |
| 11 | 373 | 4.33 | −1.5 | 0.5 |
| Av. | 533 | 4.15 | −0.9 | 0.6 |

SpH—Surface pH

### Discussion

Here it has been shown that a pH modification of REVITALIFT® Triple Power Anti-Aging Lotion will enable the generation of H₂ affected by MBMS. The results support the hypothesis that lowering the pH of a formulation below a threshold will facilitate the generation of H₂ by proper use of the MBMS. It should be noted that the MBMS lost an average of 0.9 mg. of weight when interacting with the REVITALIFT® Triple Power Anti-Aging Lotion. Thus, weight loss of the MBMS can be variable due to the different formulations evaluated.

### Example 10: Repetitive Testing of 1% Citric Acid in L′Oréal's CeraVe® Facial Moisturizing Lotion—for Generation and Transfer of Molecular Hydrogen (H₂) Facilitated by MBMS

### Background

The above Examples provided evidence that when 1% citric acid is formulated with CeraVe® Moisturizing Cream and L′Oréal REVITALIFT® Triple Power Anti-Aging Lotion, repetitive use of MBMS results in generation of H₂ over the course of more than 10-tests-when the MBMS is used to generate the H₂ on a surrogate for skin.

Here, results are shown of testing L′Oréal's Cera Ve® Facial Moisturizing Lotion for generation of H₂—using MBMS.

### Methods and Materials

CeraVe® Facial Moisturizing Lotion, was purchased on Amazon. The MBMS (14-sq. mm) was purchased ‘online’ from China. The pH measurement was taken with a calibrated (pH 4 and 7) Extech ExStik® PH100. Molecular hydrogen (H₂) was measured with a Trustlex ENH-1000 H₂ Meter adapted for skin surface measurements. A 1% anhydrous citric acid (Kraft Chemical Co.) formulation was prepared by mixing 0.3 grams of citric acid with 29.7 grams of Cera Ve Facial Moisturizing Lotion. It was stored in a 500-mL glass jar. It was used in the tests described in Table 5-below. The baseline H₂ content of CeraVe® Facial Moisturizing Lotion was 0 ppb and the pH of the acidified Lotion was 5.86.

### Procedures

Before each test, the following procedures were conducted: The weight of the Lotion in the jar was determined; The MBMS was polished with an adhesive cloth, rinsed, and dried; The weight of the MBMS was measured on an analytical balance; The MBMS was placed in the Lotion, agitated for 10-seconds, and left to incubate—for 5-minutes; The Lotion adhering to the MBMS was transferred to a tongue depressor; The sensor on the Trustlex was polished before use; The highest reading of H₂ on the Trustlex was recorded—after 15-seconds of observing readings; The weight of the Lotion remaining in the jar was recorded and the amount of Lotion removed from the jar—calculated; After removing the sample Lotion from the CeraVe® Facial Moisturizing Lotion, its weight was measured and the weight change of the MBMS determined; The pH of the Lotion on the ‘stick’ was measured.

### Results

The results are presented in Table 7.

TABLE 7

Change in

| | | | MBMS | Cream |
|---|---|---|---|---|
| Test # | H₂, ppb | SpH | Wt. (mg.) | Transfer (g.) |
| 1 | 246 | 5.86 | −1.8 | 0.7 |
| 2 | 837 | 6.16 | −0.2 | 1.1 |
| 3 | 375 | 5.73 | −1.2 | 0.8 |
| 4 | 632 | 6.70 | 0.8 | 0.8 |
| 5 | 546 | 6.09 | −0.6 | 1.0 |
| 6 | 137 | 6.47 | 0.4 | 0.9 |
| 7 | 230 | 6.59 | −0.1 | 0.7 |
| 8 | 428 | 6.56 | −0.2 | 0.7 |
| 9 | 343 | 6.52 | −0.1 | 0.7 |
| 10 | 656 | 6.56 | −0.9 | 0.9 |
| Av. | 443 | 6.32 | −0.4 | 0.8 |

### Discussion

Remarkably, the MBMS is able to affect generation of H₂ in the acidified CeraVe® Moisturizing Lotion at a pH close to 6.3. This phenomenon should allow for the MBMS to generate H₂ in a majority of skin care products in the marketplace. That is, examining the results in Table 7, it is observed that H₂ is generated when the pH is as high as 6.9. Most skin care products have a pH lower than 6.9.

### Example 11: Repetitive Testing of 1% Citric Acid in CeraVe® Resurfacing Retinol Serum for Generation and Transfer of Molecular Hydrogen (H₂) Facilitated by MBMS

### Background

The Examples above provided evidence that a 1% citric acid-when formulated with L′Oréal products, repetitively generates H₂ over the course of more than 10-tests-when the MBMS is used to generate the H₂ on a surrogate for skin. Here, results are shown of testing L′Oréal's CeraVe® Resurfacing Retinol Serum for generation of H₂ using MBMS.

### Methods and Materials

CeraVe® Resurfacing Retinol Serum was purchased on Amazon. The MBMS (14-sq. mm) was purchased ‘online’ from China. The pH measurement was taken with a calibrated (pH 4 and 7) Extech ExStik® PH100. Molecular hydrogen (H₂) was measured with a Trustlex ENH-1000 H₂ Meter adapted for skin surface measurements. A 1% anhydrous citric acid (Kraft Chemical Co.) formulation was prepared by mixing 0.3 grams of citric acid with 29.7 grams of CeraVe® Resurfacing Retinol Serum. It was stored in a 500-mL glass jar. It was used in the tests described in Table 5-below. The baseline H₂ content of CeraVe® Resurfacing Retinol Serum was 0 ppb and the pH of original serum was 5.70.

### Procedures

Before each test, the following procedures were conducted: The weight of the serum in the jar was determined; The MBMS was polished with an adhesive cloth, rinsed, and dried; The weight of the MBMS was measured on an analytical balance; The MBMS was placed in the serum, agitated for 10-seconds, and left to incubate—for 5-minutes; The serum adhering to the MBMS was transferred to a tongue depressor (‘stick’); The sensor on the Trustlex was polished before use; The highest reading of H₂ on the Trustlex was recorded—after 15-seconds of observing readings; The weight of the serum remaining in the jar was recorded and the amount of Lotion removed from the jar-calculated; After removing the sample serum from the Cera Ve® Facial Moisturizing serum, its weight was measured and the weight change of the MBMS determined; The pH of the serum on the tongue depressor—was measured.

### Results

The results are presented in Table 8.

TABLE 8

Change in

| | | | MBMS | Cream |
|---|---|---|---|---|
| Test # | H₂, ppb | SpH | Wt. (mg.) | Transfer (g.) |
| 1 | 663 | 4.80 | 0.2 | 0.5 |
| 2 | 351 | 4.25 | −1.2 | 0 |
| 3 | 359 | 4.55 | −2.0 | 0.5 |
| 4 | 446 | 4.26 | −0.9 | 0.4 |
| 5 | 502 | 4.37 | −0.2 | 0.9 |
| 6 | 360 | 4.30 | −1.4 | 0.7 |
| 7 | 186 | 4.60 | −1.1 | 0.2 |
| 8 | 486 | 4.46 | −1.5 | 0.2 |
| 9 | 775 | 4.46 | −0.9 | 0.6 |
| 10 | 426 | 4.33 | −0.1 | 0.5 |
| 11 | 629 | 4.34 | −0.9 | 0.4 |
| 12 | 502 | 4.29 | −0.7 | 0.5 |
| 13 | 337 | 5.02 | −1.4 | 0.4 |
| 14 | 280 | 4.40 | −0.2 | 0.3 |
| 15 | 23 | 4.63 | −1.4 | 0.6 |
| AV. | 422 | 4.47 | −0.91 | 0.5 |

### Discussion

This Study supports the hypothesis that with acidic modification of existing formulations, the MBMS can affect generation of H₂ in that product. Also of interest, the observation that the MBMS lost an average of 0.9 mg. of weight per test. It is of interest to determine if loss of weight of the MBMS takes place as an inverse function of pH or as a function of citric acid concentration.

### Example 12: Effect of pH on Generation of H₂—Using CeraVe® Moisturizing Cream Activated by a ZNANODE

### Background

In the previous Examples, it has been demonstrated that an MBMS, when incubated with a qualified skin care formulation, can transfer an H₂-containing formulation to the skin. Here, studies are presented on:

(1) The H₂ generation-affected by a zinc base metal sheet (ZNANODE), in Cera Ve® Moisturizing Cream.

(2) The effect altering the pH of CeraVe® MC on ZNANODE generation of H₂.

### Materials and Methods

CeraVe® Moisturizing Cream was purchased on Amazon. Anhydrous citric acid was purchased from Kraft Chemical Co. The ZNANODE (40 sq. cm) was purchased ‘online’ from a Chinese source.

The various citric acid—CeraVe® Moisturizing Cream formulations were prepared by adding different percentages of citric acid to bring the CeraVe® Moisturizing Cream to 30-grams—stored in 50-mL glass jars.

The pH measurement was taken with a calibrated (pH 4 and 7) Extech ExStik® PH100. Molecular hydrogen (H₂) was measured with a Trustlex ENH-1000 H₂ Meter adapted for skin surface measurements.

### Procedures and Results

The ZNANODE was immersed in Cera Ve® Moisturizing Cream—for 5-minutes.

The ZNANODE, with cream adhered to it, was removed from its jar and the cream on the ZNANODE was rubbed for 30-seconds onto a wooden tongue depressor. The cream on the tongue depressor surface was then evaluated for H₂—with the Trustlex H₂ Meter.

### Results

The results are delineated in Table 9.

TABLE 9
H₂ Generation by ZNANODE in Acidified CeraVe MC

| % | | | Change in | |
|---|---|---|---|---|
| Citric | | | ZNANODE | Cream |
| Acid | SpH | H₂, ppb | Wt. (mg.) | Transfer (g.) |
| 0.00 | 5.04 | 0 | −1.7 | 0.6 |
| 0.005 | 3.89 | 95 | −0.3 | |
| 0.010 | 3.55 | 135 | −1.4 | 0.5 |
| 0.025 | 3.24 | 576 | −0.7 | 0.6 |
| 0.050 | 3.12 | 655 | ND | 0.7 |
| 0.100 | 2.69 | 628 | −2.0 | 0.6 |
| 0.200 | 2.51 | 649 | −1.0 | 0.6 |
| 0.300 | 2.40 | 615 | −0.2 | 0.4 |
| 0.400 | 2.29 | 611 | −0.1 | 0.9 |

### Discussion

Unlike the MBMS, the ZNANODE has a narrower pH range for appreciable activation of the Cream in generating H₂. That is, at a pH higher than 3.24, H₂—generation with ZNANODE drops off and by pH 5—ZNANODE generated zero H₂. In contrast, it has been previously demonstrated that the MBMS can activate a formulation to generate H₂ at a pH as high as 6.6 (Example 11). Therefore, the MBMS is likely to be able to generate H₂ in more marketed products—as compared to the ZNANODE.

### Example 13: Effect of pH on Generation of H₂-Using CeraVe® Moisturizing Cream Activated by FEANODE

### Background

In the previous Examples, it has been demonstrated that an MBMS and a ZNANODE, when incubated with a qualified skin care formulation, can generate, and transfer an H₂-containing formulation to the skin. Here, studies are presented on:

(1) The H₂ generation—affected by an iron base surface (FEANODE), in CeraVe® Moisturizing Cream.

(2) The effect of altering the pH of CeraVe® MC on FEANODE generation of H₂.

### Materials and Methods

CeraVe® Moisturizing Cream was purchased on Amazon. Anhydrous citric acid was purchased from Kraft Chemical Co. The FEANODE (40 sq. cm) was purchased ‘online’ from a Chinese source.

The various citric acid—CeraVe® Moisturizing Cream formulations were prepared by adding different percentages of citric acid to bring the CeraVe® Moisturizing Cream to 30-grams—stored in 50-mL glass jars. For increasing the pH above that of unadulterated CeraVe® M C, triethanolamine was added and mixed into the Cera Ve® M C.

The pH measurement was taken with a calibrated (pH 4 and 7) Extech ExStik® PH100. Molecular hydrogen (H₂) was measured with a Trustlex ENH-1000 H₂ Meter adapted for skin surface measurements.

### Procedures and Results

The FEANODE was immersed in CeraVe® Moisturizing Cream—for 5-minutes. The FEANODE, with cream adhered to it, was removed from its jar and the cream on the FEANODE was rubbed for 30-seconds onto a wooden tongue depressor (surrogate for skin). The cream on the tongue depressor surface was then evaluated for H₂—with the Trustlex H₂ Meter.

### Results

The results are displayed in Table 11.

TABLE 11
Example 13

| | | FEANODE | Cream | % w/w | |
|---|---|---|---|---|---|
| | H₂, | Wt. | Trans. | Citric | % w/w |
| SpH | ppb | Ch.(mg.) | (g) | acid | TEA* |
| 2.98 | 377 | 1.6 | −0.9 | 0.8 | 0.0 |
| 3.04 | 286 | −1.1 | −0.7 | 0.4 | 0.0 |
| 3.41 | 135 | −0.4 | −0.6 | 0.2 | 0.0 |
| 3.64 | 243 | 0.4 | −0.7 | 0.1 | 0.0 |
| 4.07 | 135 | 0.3 | −0.8 | 0.05 | 0.0 |
| 5.76 | 138 | 0 | −0.5 | 0.0 | 0.0 |
| 6.49 | 183 | 2.7 | −0.9 | 0.0 | 0.25 |
| 7.42 | 144 | 1.9 | 0.9 | 0.0 | 0.5 |
| 7.77 | 125 | 0.5 | −0.7 | 0.0 | 1.0 |

*TEA Triethanolamine

### Discussion

Like the MBMS and the ZNANODE, the FEANODE affects the generation of H₂ in a qualified skin care formulation. Unlike the MBMS and the ZNANODE, the FEANODE generates low level H₂-over a wider range of pH values (i.e., pH 3-7.7). For practical purposes, H₂ generation at pHs higher than 4 would not be adequate.

### Example 14: Some Devices for H₂-Activation, Application and Massaging Formulations into Facial and Body Skin

### Background

In previous Examples, it has been demonstrated that application and massaging of a qualified skin care formulation, into the skin of a subject-using either a MBMS, or a ZNANODE or a FEANODE results in generation of H₂-on the surface of the skin or a surrogate for skin. Here, some Examples are presented of H₂-generating devices that can be used for this purpose.

### Materials

H₂ activating devices shown in the figures above can be made of either magnesium, zinc, or iron. They can be made of high-quality metal by a contract fabricator of such products. The Vibrating Massage Head or an ultrasonic head of the illustration of the Facial Massager, displayed in FIG. 3 can be made of either a MBMS, or a ZNANODE or a FEANODE. It can be retrofitted by a contract manufacturer.

### Results and Discussion

FIG. 1 shows a mechanical device, made with pure magnesium or pure zinc or pure iron. These devices, to be referred to as H₂-activating cosmetic spatulas, are multi-purpose, in that they can be used for one or all of the following purposes: Mix ingredients into the skin care formulation of interest; Generate H₂—in the skin care formulation; Apply a skin care formulation to the skin; Apply and massage an H₂-activated skin care formula into skin; Massage a qualified skin care formulation into the skin-thereby generating H₂ on the surface of the skin.

FIG. 3 displays an electronic facial massager, that when retrofitted, with a vibrating massage head composed of pure magnesium or pure zinc or pure iron will generate H₂ on the surface of the skin—when massaging a qualified skin care formulation into the skin. The electronic massager or a corresponding ultrasonic massager—can have other features including: Red Light for treatment of aging skin; Blue Light for treatment of acne-like blemishes; Vibrating massage head for massaging formulations into skin; Temperature heating for enhanced penetration; Speed control; Generates H₂—while removing make-up.

### Example 15: One Hundred Repeat Testing of CeraVe® MC—for Generation and Transfer of Molecular Hydrogen (H₂)-Enabled by an MBMS

### Background

Previous Examples have indicated that the MBSM can be re-used up to 30-times, with a qualified skin care formulation, to generate H₂ on the surrogate for the skin surface. In these previous studies, the MBMS was polished before re-use.

In contrast, in this Study, the MBMS was polished only after the generation of H₂ with massaging the Cream on the skin reached a generation of H₂-below 100 ppb. A total of one hundred consecutive tests were conducted. The interpretation of these results will provide advice to the consumer as to how often they should polish their MBMS.

### Methods and Materials

CeraVe® Moisturizing Cream was purchased on Amazon. The MBMS (492.6-sq. cm) was purchased ‘online’ from China.

Molecular hydrogen (H₂) was measured with a Trustlex ENH-1000 H₂ Meter adapted for skin surface measurements.

### Procedures

Before each test, the following procedures were conducted: The weight of the Cream in the jar was determined; The MBMS washed and dried (not polished with an abrasive instrument); The weight of the MBMS was measured on an analytical balance; A tongue depressor was used to remove two grams of Cream from the Jar; The Cream remained on the tongue depressor; The sensor on the Trustlex was polished before use; The Trustlex was moved back and forth on the tongue depressor; The highest reading of H₂ on the Trustlex was recorded-after 15-seconds of observing readings: After removing the Cream from the MBMS, its weight was measured and the weight change of the MBMS, determined.

### Results

The results are presented in Table 12.

TABLE 12
100 Repeat Generations of H₂-CeraVe ® M
C- on a Surrogate for Skin

| | | | Change in | |
|---|---|---|---|---|
| | | | MBMS | |
| | Test # | H₂, ppb | Wt. (mg.) | Comment |
| | 1 | 325 | 0.0 | |
| | 2 | 514 | −0.1 | |
| | 3 | 522 | −0.5 | |
| | 4 | 550 | −0.9 | |
| | 5 | 553 | −1.1 | |
| | 6 | 394 | −0.1 | |
| | 7 | 475 | 0.0 | |
| | 8 | 425 | 0.7 | |
| | 9 | 526 | 0.3 | |
| | 10 | 458 | 0.7 | |
| | 11 | 373 | 0.2 | |
| | 12 | 455 | −0.3 | |
| | 13 | 441 | 0.7 | |
| | 14 | 462 | −0.6 | |
| | 15 | 530 | 0.3 | |
| | 16 | 336 | 0.9 | |
| | 17 | 451 | −0.7 | |
| | 18 | 410 | 1.5 | |
| | 19 | 412 | −1.5 | |
| | 20 | 528 | −0.4 | |
| | 21 | 369 | 0.2 | |
| | 22 | 334 | 0.3 | |
| | 23 | 255 | 0.0 | |
| | 24 | 430 | 0.3 | |
| | 25 | 390 | −0.1 | |
| | 26 | 388 | 0.2 | |
| | 27 | 359 | 1.0 | |
| | 28 | 441 | −0.9 | |
| | 29 | 266 | −0.8 | |
| | 30 | 255 | 0.7 | |
| | 31 | 172 | −0.13 | |
| | 32 | 263 | −0.5 | |
| | 33 | 45 | 0.6 | Polished MBMS |
| | 34 | 504 | 1.2 | |
| | 35 | 349 | 1.7 | |
| | 36 | 333 | 0.7 | |
| | 37 | 328 | −0.1 | |
| | 38 | 729 | 1.3 | |
| | 39 | 278 | 0.0 | |
| | 40 | 606 | −0.1 | |
| | 41 | 316 | −0.9 | |
| | 42 | 355 | 0.5 | |
| | 43 | 280 | −0.8 | |
| | 44 | 438 | −0.4 | |
| | 45 | 408 | −0.4 | |
| | 46 | 568 | −0.1 | |
| | 47 | 555 | 1.9 | |
| | 48 | 337 | −1.9 | |
| | 49 | 236 | 0.1 | |
| | 50 | 219 | 2.5 | |
| | 51 | 143 | 1.7 | |
| | 52 | 117 | −0.5 | |
| | 53 | 151 | −1.2 | |
| | 54 | 179 | −3.3 | |
| | 55 | 211 | −1.2 | |
| | 56 | 319 | 1.5 | |
| | 57 | 317 | 0.4 | |
| | 58 | 460 | −0.14 | |
| | 59 | 418 | 0.5 | |
| | 60 | 275 | 0.6 | |
| | 61 | 346 | −2.1 | |
| | 62 | 325 | 1.5 | |
| | 63 | 278 | −1.6 | |
| | 64 | 332 | 2.2 | |
| | 65 | 373 | 1.0 | |
| | 66 | 319 | −0.8 | |
| | 67 | 486 | −0.6 | |
| | 68 | 441 | 0.0 | |
| | 69 | 141 | 0.3 | |
| | 70 | 266 | −0.1 | |
| | 71 | 219 | −0.6 | |
| | 72 | 345 | 0.0 | |
| | 73 | 333 | −0.9 | |
| | 74 | 204 | 0.2 | |
| | 75 | 129 | 1.6 | |
| | 76 | 331 | −0.1 | |
| | 77 | 199 | −0.2 | |
| | 78 | 152 | −0.2 | |
| | 79 | 335 | −0.1 | |
| | 80 | 495 | −0.6 | |
| | 81 | 321 | 0.7 | |
| | 82 | 192 | 1.2 | |
| | 83 | 174 | −1.4 | |
| | 84 | 238 | 1.8 | |
| | 85 | 169 | 0.7 | |
| | 86 | 229 | 0.1 | |
| | 87 | 251 | 1.0 | |
| | 88 | 172 | −0.5 | |
| | 89 | 188 | 0.3 | |
| | 90 | 107 | −4.3 | |
| | 91 | 194 | 0.1 | |
| | 92 | 241 | 0.2 | |
| | 93 | 74 | 0.3 | Polished MBMS |
| | 94 | 978 | 0.6 | |
| | 95 | 574 | 0.8 | |
| | 96 | 533 | −0.3 | |
| | 97 | 628 | 0.6 | |
| | 98 | 438 | 0.4 | |
| | 99 | 726 | 0.2 | |
| | 100 | 449 | 0.2 | |
| Av. | | 355 | 0.1 | |

### Discussion

This Study was conducted to determine if the MBMS can be used repeatedly, i.e., one hundred times without losing its potential to catalyze Ha generation in a qualified skin care formulation. It was found that Ha production does diminish with use of the MSMBS—but is 100% restored by polishing with an abrasive instrument. Thus, users of the MBMS can often polish the MBMS i.e., after 10—uses.

A surprising finding is the observation that taking the average of the 100-weight changes of the MSBS, there is no net weight loss. This Study was conducted using unadulterated CeraVe® M C that has a pH of about 5.1. This result contrasts with the other Studies (see Tables 5, 6 and 8 done—with citric acid acidified products—where average 0.6 to 0.9 mg. net weight loss took place. Citric acid may cause a small ‘leaching out’ of magnesium from the MBMS probably forming magnesium citrate. There may be ‘wisdom’ in developing qualified products that do not leach material out of BMS.

While the flowcharts presented for this technology may imply a specific order of execution, the order of execution may differ from what is illustrated. For example, the order of two more blocks may be rearranged relative to the order shown. Further, two or more blocks shown in succession may be executed in parallel or with partial parallelization. In some configurations, one or more blocks shown in the flow chart may be omitted or skipped. Any number of counters, state variables, warning semaphores, or messages might be added to the logical flow for purposes of enhanced utility, accounting, performance, measurement, troubleshooting or for similar reasons.

Reference was made to the examples illustrated in the drawings and specific language was used herein to describe the same. It will nevertheless be understood that no limitation of the scope of the technology is thereby intended. Alterations and further modifications of the features illustrated herein and additional applications of the examples as illustrated herein are to be considered within the scope of the description.

Furthermore, the described features, structures, or characteristics may be combined in any suitable manner in one or more examples. In the preceding description, numerous specific details were provided, such as examples of various configurations to provide a thorough understanding of examples of the described technology. It will be recognized, however, that the technology may be practiced without one or more of the specific details, or with other methods, components, devices, etc. In other instances, well-known structures or operations are not shown or described in detail to avoid obscuring aspects of the technology.

Although the subject matter has been described in language specific to structural features and/or operations, it is to be understood that the subject matter defined in the appended claims is not necessarily limited to the specific features and operations described above. Rather, the specific features and acts described above are disclosed as example forms of implementing the claims. Numerous modifications and alternative arrangements may be devised without departing from the spirit and scope of the described technology.

What is claimed is:

1. A method of generating hydrogen gas on skin, comprising:
contacting a solid base metal surface with an aqueous skin care formulation to coat the solid base metal surface with the aqueous skin care formulation forming a formulation-coated solid base metal surface;
catalytically converting a portion of the aqueous skin care formulation to molecular hydrogen (H₂) at the solid base metal surface; and
massaging skin using the formulation-coated solid base metal surface to generate H₂ on the skin.

2. The method of claim 1, wherein the solid base metal surface is composed of at least 90.0% magnesium metal.

3. The method of claim 1, wherein the solid base metal surface is composed of at least 99.995% magnesium metal.

4. The method of claim 1, wherein the solid base metal surface is composed of at least 80.0% zinc metal.

5. The method of claim 1, wherein the solid base metal surface is composed of at least 99.99% zinc metal.

6. The method of claim 1, wherein the solid base metal surface is composed of at least 90.0% iron metal.

7. The method of claim 1, wherein the solid base metal surface is composed of at least 99.99% iron metal.

8. The method of claim 1, wherein a thickness of the solid base metal surface is at least 0.10 mm and no more than 30.0 mm and wherein the solid base metal surface is in the form of a foil, a metal rod, a spatula, a cylinder, a sphere, or a combination thereof.

9. The method of claim 1, wherein the aqueous skin care formulation has a pH lower than 6.9 and not lower than 2.0.

10. The method of claim 1, wherein the aqueous skin care formulation has a pH lower than 4.0 and not lower than 2.0.

11. The method of claim 1, wherein the aqueous skin care formulation is in the form of a hydrogel, cream, lotion, serum, spray, foam, or combination thereof.

12. The method of claim 1, further comprising adding a USP grade organic acid.

13. The method of claim 12, wherein the organic acid is selected from the group consisting of: citric acid, malic acid, adipic acid, phosphoric acid, and combinations thereof to adjust a pH of the aqueous skin care formulation.

14. The method of claim 1, wherein the solid base metal surface is placed in a container with the aqueous skin care formulation and allowed to remain there for 1 second to 60 minutes to generate 100-1400 ppb H₂ in the aqueous skin care formulation on solid base metal surface.

15. The method of claim 1, wherein the aqueous skin care formulation is first placed on a designated site on the skin—followed by massaging that skin site with the solid base metal surface to generate H₂ on the skin surface.

16. The method of claim 1, wherein the solid base metal surface is used to massage the aqueous skin care formulation into the skin for one second to 1-hour to generate H₂ on the surface of the skin.

17. The method of claim 1, further comprising polishing the solid base metal surface with an abrasive instrument before contacting the solid base metal surface with the aqueous skin care formulation.

18. The method of claim 17, wherein the abrasive instrument is steel wool, sandpaper, or a stainless-steel brush capable of removing oxidized materials on the solid base metal surface.

19. The method of claim 1, wherein the aqueous skin care formulation further comprises a nitrite salt such that the massaging the skin will also generate nitric oxide in the skin.

20. An electronic or ultrasonic facial massager comprising a vibrating massage head comprising a solid base metal surface composed of at least 90% magnesium or zinc or iron configured to generate H₂ on the surface of skin when massaging an aqueous skin care formulation into the skin.

21. The massager of claim 20, further comprising at least one of: lights for red light therapy, lights for blue light therapy, a temperature control, and a speed control.

22. The massager of claim 20, wherein the solid base metal surface comprises a magnesium or zinc or iron foil having a thickness of 0.05 mm to 10 mm adhered to the massage head.

23. A kit comprising instructions to perform a method according to claim 1 and one or more of: a solid base metal surface spatula, an electronic or ultrasonic skin massager with a massage head comprising a solid base metal surface, an aqueous skin care formulation, an abrasive instrument for polishing the solid base metal surface, a packet containing an acid for acidification of an aqueous skin care formulation, and a pH paper for determining the pH of an aqueous skin care formulation.

24. The kit of claim 23, wherein the kit comprises the aqueous skin care formulation and the solid base metal spatula or the massager, and wherein the aqueous skin care formulation has a pH from 2.0 to 6.5 such that the aqueous skin care formulation is capable of forming H₂ when contacted with the solid base metal surface.
